(19) United States
(12) Patent Application Publication
(10) Pub. No.: US 2009/0113609 A1
(43) Pub. Date: May 7, 2009

(54) Attachment For Swimming Goggles

(76) Inventor: Joseph Allessie, Ringwood, NJ (US)

Correspondence Address: WEST & ASSOCIATES, A PC, 2815 MITCHELL DRIVE, SUITE 209, WALNUT CREEK, CA, 94598, US

(21) Appl. No.: 12/261,904

(22) Filed: Oct. 30, 2008

Related U.S. Application Data
(60) Provisional application No. 60/983,686, filed on Oct. 30, 2007.

(51) Int. Cl. A61F 9/02

(52) U.S. Cl. 2/452; 2/15

(57) ABSTRACT

A sporting-goods device capable of removably attaching to eyewear, specifically swimming goggles, and another object to prevent loss of the eyewear.

### CLAIM OF PRIORITY

This Application claims priority under 35 U.S.C. § 119(e) from earlier filed U.S. Provisional Application Ser. No. 60/983,686, filed Oct. 30, 2007, by Joseph Allessie, the entirety of which is incorporated herein by reference.

### BACKGROUND

1. Field of the Invention

The present disclosure relates to the field of sporting goods, specifically an accessory to prevent loss of swimming goggles.

2. Background

Recreational and professional swimmers alike use swimming goggles to both protect their eyes from chlorinated water and to be able to see clearly under water. However, many swimmers often find themselves in need of a new pair because they mistakenly leave their goggles on the edge of the pool, on a lounge chair, in a locker room, or because they sink and cannot be located.

Replacing lost goggles can be expensive, especially with children who are notorious for losing goggles, for professional swimmers who often buy high-end, high-priced goggles, or for swimmers who buy custom corrective lens goggles. It is therefore important to have a lightweight goggle accessory that can removably attach to both a pair of goggles and another object, such as an arm, a pool bag, or a keychain. It is also important to keep goggles afloat to prevent sinking. This type of device would keep a swimmer's goggles either on his or her body or together with other personal items when not in use, thereby decreasing the chance of loss or inadvertent damage. This type of device could be used with other kinds of eyewear, such as sunglasses or reading glasses, to prevent loss of those items as well.

Eyeglass retention devices currently exist on the market, such as those sold by Croakies®, but those devices are not suitable for use with goggles. Additionally, although a goggle retaining device is described in U.S. Pat. No. 5,987,650, that device is for attachment of goggles to a swimsuit, is not removable from the swimsuit, and therefore cannot be used with other objects, such as a tote bag.

What is needed is a lightweight device that can removably attach to both a pair of goggles or other eyewear and the owner's body or another object to prevent loss.

### BRIEF DESCRIPTION OF THE DRAWINGS

FIG. 1 depicts a perspective view of an embodiment of the present device.

FIG. 1A depicts a perspective view of another embodiment of the present device.

FIG. 1B depicts a perspective view of yet another embodiment of the present device.

FIG. 1C depicts a perspective view of yet another embodiment of the present device.

FIG. 2 depicts a perspective view of an embodiment of the present device in an in-use configuration.

### DETAILED DESCRIPTION

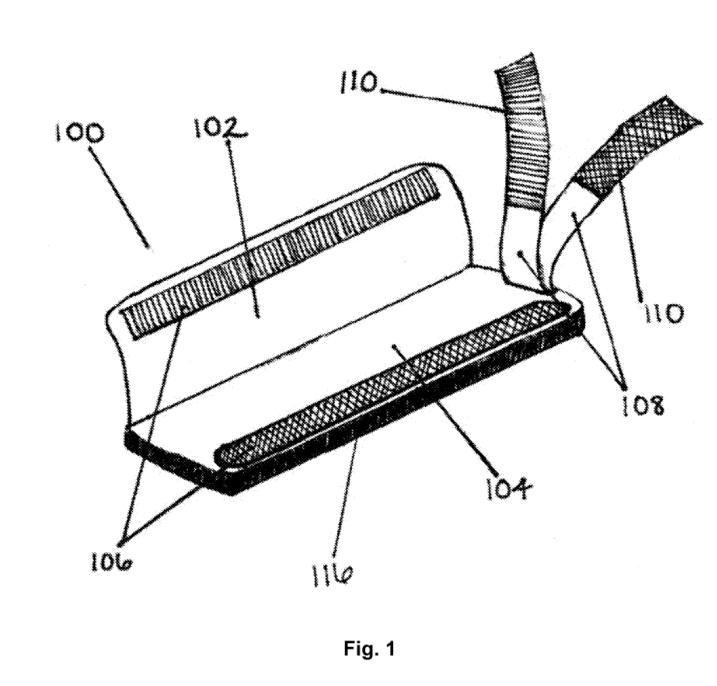

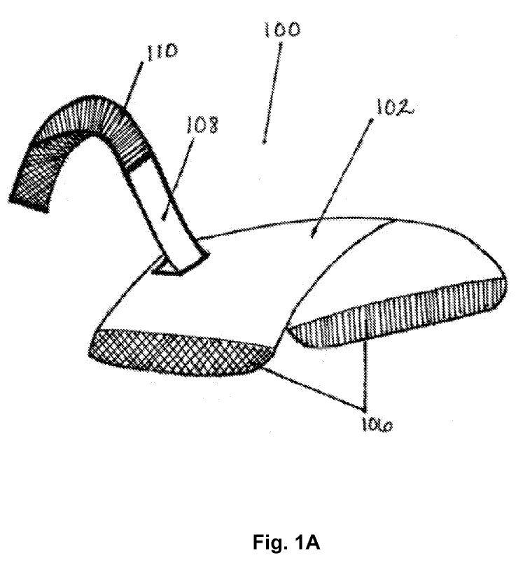

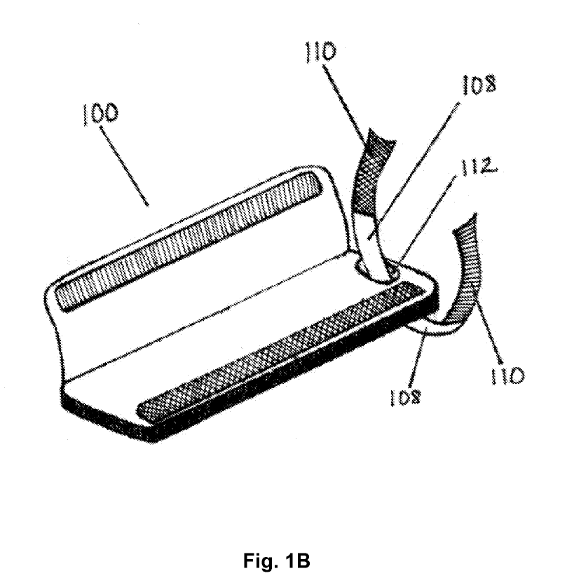

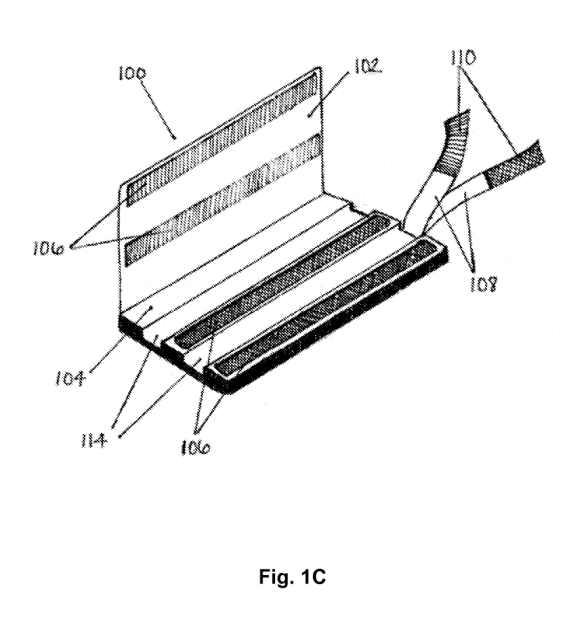

FIG. 1 shows a perspective view of an embodiment of the present device 100. At least one edge of the lower surface of a first substantially planar member 102 can be connected to at least one edge of the upper surface of a second substantially planar member 104. The lower surface of said first substantially planar member 102 and the upper surface of said second substantially planar member 104 can each be connected to at least one fastening region 106. Said fastening regions 106 can be paired to mate with each other, such that said first and second substantially planar members 102 and 104 can removably attach to each other. Fastening regions 106 can be in the form of strips covering a small portion of each of said first and second substantially planar members 102 and 104, as shown in FIGS. 1-IC, or can cover the entire surface area of at least one of said first and second substantially planar members 102 and 104.

At least one of said first and second substantially planar members 102 and 104 can be coupled with at least one elongated flexible member 108. In FIG. 1, elongated flexible members 108 are connected substantially close to a short edge of a second substantially planar member 104. In another embodiment, as shown in FIG. 1B, a second substantially planar member 104 can have an aperture 112 adapted to accept at least one elongated flexible member 108. In alternate embodiments, at least one elongated flexible member 108 can be coupled with any other known and/or convenient location on or through at least one of said first or second substantially planar members 102 and 104.

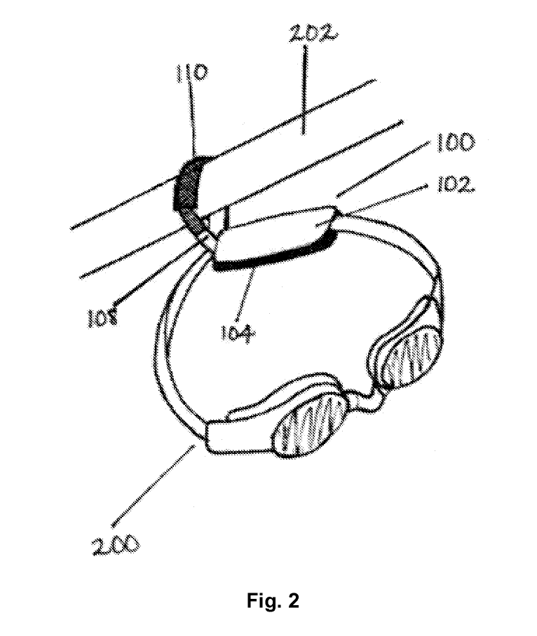

As shown in FIG. 1C, at least one of said first and second substantially planar members 102 and 104 can have at least one channel 114 running through it adapted to accommodate the strap of a pair of goggles and/or at least one elongated flexible member 108 and temporarily secure said strap and/or elongated flexible member 108 when substantially planar members 102 and 104 are removably attached to each other via said fastening regions 106 (see FIG. 2). FIG. 1C shows a device 100 comprising two channels 114 and two sets of fastening regions 106 on the surfaces of substantially planar members 102 and 104, such that a user can selectively cover or expose one or both channels 114 with a first substantially planar member 102. In this embodiment, a user can keep the strap of a pair of goggles secured within device 100 while selectively exposing at least one elongated flexible member 108. In other embodiments, a single channel 114 can be adapted to accommodate both a goggle strap and at least one elongated flexible member 108 at the same time.

At least one side of said elongated flexible member 108 can be coupled with at least one set of complementary fastening components 110. As shown in FIG. 1, two elongated flexible members 108 can be coupled with a set of complementary fastening components 110 such that said elongated flexible members 108 can removably attach to each other. As shown in FIG. 1A, in another embodiment device 100 can have one elongated flexible member 108, both sides of which can be coupled with a set of complementary fastening components 110 such that the ends of said elongated flexible member 108 can removably attach to each other.

FIG. 1 shows said fastening regions 106 as being connected to said first and second substantially planar members 102 and 104 along an edge opposite the edge where the substantially planar members 102 and 104 are connected to each other. In an alternate embodiment, at least one fastening region 106 can be integral with the material of at least one of said first and second substantially planar members 102 and 104. In other embodiments said fastening regions 106 can be connected at any other known and/or convenient location on said first and second substantially planar members 102 and 104.

As shown in FIG. 1A, an alternate embodiment of the device 100 can have just one substantially planar member 102 having an upper surface and a lower surface. Said substantially planar member 102 can connected to at least one fastening region 106, such that one edge of the upper surface of a substantially planar member 102 can removably attach to the opposite edge of the lower surface of a substantially planar member 102. In some embodiments, at least two edges of each of the upper and lower surfaces of a substantially planar member 102 can be connected to fastening regions 106, such that opposite edges of the same surface can also removably attach to each other.

In FIG. 1, said first and second substantially planar members 102 and 104 are rectangular. In alternate embodiments, said first and second substantially planar members 102 and 104 can have any other known and/or convenient geometry. In FIG. 1, the first and second substantially planar members 102 and 104 are made of neoprene. In alternate embodiments, at least one of said first and second substantially planar members 102 and 104 can be made of any other known and/or convenient buoyant material, such that the entire device 100 can float in a body of water and can keep afloat a pair of goggles. In other embodiments, at least one of said first and second substantially planar members 102 and 104 can be made of or can be connected to an inflatable bladder 116, whereby a user can selectively inflate the bladder 116 by blowing air into it, thereby making the device 100 buoyant. Said inflatable bladder 116 can be a single cell or can be comprised of a plurality of cells that inflate simultaneously. In yet other embodiments, said bladder 116 can be pre-filled either wholly or partially with a water-buoyant gas or liquid and sealed.

Said fastening regions 106 and set of complementary fastening components 110 can be hook and loop, snaps, protrusion and aperture, or any other known and/or convenient fastening mechanisms. At least one of said first and second substantially planar members 102 and 104 can be connected to a piece of fluorescent material such that the device 100 can be easily spotted from a distance or in the dark. In alternate embodiments, at least one of said first and second substantially planar members 102 and 104 can be made of or integral with a piece of fluorescent material. A first substantially planar member 102 can also be a different color than a second substantially planar member 104. In use, each device 100 of a set of devices 100 can have distinguishing colors and/or markings such that users can identify which device 100 belongs to each owner or each pair of goggles.

Referring to FIG. 2, in use a user wraps first and second substantially planar members 102 and 104 around a strap of a pair of goggles 200. A user can then removably attach one edge of the lower surface of a first substantially planar member 102 to the upper surface of a second substantially planar member 104 via fastening regions 106. At this stage, the device 100 can be removably attached to the strap of a pair of goggles 200. A user can then wrap elongated flexible members 108 around a convenient object 202, as shown in FIG. 2, or a part of the user's body. A user can then removably attach said elongated flexible members 108 to each other via a set of complementary fastening components 110, such that the device 100 is removably attached to both a pair of goggles 200 and a convenient object 202.

Alternatively, a user can tuck elongated flexible members 108 inside said first and second substantially planar members 102 and 104 while said substantially planar members 102 and 104 are wrapped around the strap of a pair of goggles 200. In this manner, a user does not have to attach the elongated flexible members 108 to a convenient object 202. Although a pair of goggles 200 would not be attached to a convenient object 202, the device 100 could still keep a pair of goggles 200 afloat if they became separated from a user and landed in a body of water.

Although the invention has been described in conjunction with specific embodiments thereof, it is evident that many alternatives, modifications and variations will be apparent to those skilled in the art. Accordingly, the invention as described and hereinafter claimed is intended to embrace all such alternatives, modifications and variations that fall within the spirit and broad scope of the appended claims.

What is claimed is:

1. An eyewear retaining device, comprising:
a first substantially planar member having an upper surface and a lower surface;
a second substantially planar member having an upper surface and a lower surface;
at least one edge of said lower surface of said first substantially planar member being connected to at least one edge of said upper surface of said second substantially planar member;
said lower surface of said first substantially planar member and said upper surface of said second substantially planar member each being connected to at least one fastening region, such that said fastening regions can be paired to mate with each other and said first and second substantially planar members can removably attach to each other;
at least one of said first and second substantially planar members being coupled with one end of at least one elongated flexible member;
said at least one elongated flexible member having a first side and a second side;
at least one of said first and second sides of said at least one elongated flexible member being connected to a set of complementary fastening components.

2. The device of claim 1, wherein at least one of said first and second substantially planar members has at least one channel adapted to accommodate the strap of a pair of goggles and/or said at least one elongated flexible member.

3. The device of claim 1, wherein at least one of said first and second substantially planar members has an aperture for receiving said at least one elongated flexible member.

4. The device of claim 1, wherein a second elongated flexible member can removably couple to said at least one elongated flexible member via said second set of complementary fastening components.

5. The device of claim 1, wherein at least one of said first and second substantially planar members is made of buoyant material.

6. The device of claim 5, wherein said buoyant material is neoprene.

7. The device of claim 1, wherein at least one of said first and second substantially planar members is connected to a piece of fluorescent material.

8. The device of claim 1, wherein at least one of said first and second substantially planar members is made of or integral with a piece of fluorescent material.

9. The device of claim 1, wherein at least one of said first and second substantially planar members is made of or connected to a selectively inflatable bladder.

10. The device of claim 1, wherein at least one of said first and second substantially planar members is made of or connected to a sealed bladder filled with a water-buoyant gas or liquid.

11. An eyewear retaining device, comprising:
a substantially planar member having an upper surface and a lower surface;
at least one edge of each of said upper and lower surfaces of a substantially planar member being connected to a first set of complementary fastening components, such that one edge of said upper surface can removably attach to the opposite edge of either said upper surface or said lower surface;
at least one elongated flexible member having a first side and a second side;
at least one of said first and second sides of said at least one elongated flexible member being connected to a second set of complementary fastening components.

12. The device of claim 11, wherein said substantially planar member has an aperture for receiving said at least one elongated flexible member.

13. The device of claim 11, wherein a second elongated flexible member can removably couple to said at least one elongated flexible member via said second set of complementary fastening components.

14. The device of claim 11, wherein at least one of said first and second substantially planar members is made of buoyant material.

15. The device of claim 14, wherein said buoyant material is neoprene.

16. The device of claim 11, wherein said substantially planar member is connected to a piece of fluorescent material.

17. The device of claim 11, wherein said substantially planar member is connected to a selectively inflatable bladder.

18. The device of claim 11, wherein said substantially planar member is connected to a sealed bladder filled with a water-buoyant gas or liquid.
